(19) United States
(12) Patent Application Publication
(10) Pub. No.: US 2004/0111133 A1
(43) Pub. Date: Jun. 10, 2004

(54) LED illuminator

(75) Inventors: John C. Huculak, Mission Viejo, CA (US); James W. Phillips, Fountain Valley, CA (US)

Correspondence Address: Alan R. Thiele, JENKENS & GILCHRIST, P.C., Suite 3200, 1445 Ross Avenue, Dallas, TX, 75202-2799, US

(73) Assignee: Alcon, Inc.

(21) Appl. No.: 10/671,150

(22) Filed: Sep. 25, 2003

Related U.S. Application Data
(60) Provisional application No. 60/432,133, filed on Dec. 10, 2002.

(51) Int. Cl. A61N 1/00

(52) U.S. Cl. 607/88

(57) ABSTRACT

An illumination system to be used within the body of an animal includes a cannula mounted on a handle. On the end of the cannula to be inserted within the animal is a light emitting diode. Either a wired or a wireless system controls the illumination of the LED.

### PRIORITY

This application claims priority from U.S. Provisional Patent Application Serial No. 60/432,133 filed Dec. 10, 2002.

### FIELD

The present invention pertains to illuminators used for providing visibility to interior spaces within the body of a human being or an animal (hereinafter “animal”).

### BACKGROUND

Advances in fiberoptic technology in recent years have had a major impact on the ability of physicians, to include veterinarians, to either examine or perform procedures within the body of an animal. An example of a procedure within the body of an animal is an operation to either repair a defect or to treat a disease within the eye.

One example of a prior art illuminator is marketed by the assignee of the instant application under the Accurus® brand. The Accurus® illuminator utilizes a halogen lamp located at a console to generate light. The light at the console is focused and launched into a disposable fiberoptic probe. The disposable fiberoptic probe provides a path for the light produced by the halogen lamp as the light travels from the console into the posterior chamber of the patient's eye, where the light is used to illuminate an area of interest; for example, during vitreoretinal surgery.

Prior art illuminators used in surgery in the posterior of an eye consist of relatively large, inefficient light sources. These prior art illuminators typically require special power sources and generate wasted heat. The allocation of electrical power, cooling, and space within a console to support prior art illuminators adds to the cost of the console. In addition, prior art console-supported illuminators require routine changing of light bulbs.

Accordingly, there remains a need in the art for a low cost illuminator which makes efficient use of electrical power, generates a minimal amount of heat, and does not require routine changing of light bulbs.

### SUMMARY OF THE INVENTION

The low cost illuminator of the present invention makes efficient use of electrical power, generates a minimal amount of heat, and does not require routine changing of light bulbs.

The disclosed illuminator includes a handle, a cannula extending from the handle, and a light emitting diode (LED) mounted on the end of the cannula. Control over the operation of the LED is effected either by a wire connection to a console containing the required electronic componentry or a wireless connection between a transmitter in a console and a receiver in the handle portion of the illuminator.

The disclosed LED illuminator provides the following advantages:

1. The single use handheld probe, including the handle, the cannula, and the LED, of the illuminator is disposable, inexpensive, and easy to manufacture.

2. The console requires less interior space to support the illumination function. The elimination of the need for the console to contain an incandescent or arc lamp lowers the temperature (cooling) requirements of the console and makes the overall design of the system simpler, less expensive, and more reliable.

3. Illumination of interior spaces within the body of an animal with different lighting characteristics is possible simply by changing disposable probes containing different LED's. When different lighting is needed for different applications, different LED's may be made a part of the single use handheld probe.

4. LED's are energy efficient and produce very little heat. Therefore, the overall operating temperature of the equipment (console and handheld probe) will be much lower.

5. Wires (if used) are more flexible than fiberoptic cable. This flexibility affords more manageable cable maintenance in the sterile field during setup and during use.

6. The wireless battery operated configuration eliminates the requirement for setup and the need for cable management which provides the maximum maneuverability for the physician or veterinarian.

### BRIEF DESCRIPTION OF THE DRAWING FIGURES

A better understanding of the LED illuminator of the present invention may be had by reference to the drawing figures, wherein:

FIG. 1 is a schematic view of a first embodiment of the present invention;

FIG. 2 is a schematic view of a second, wireless embodiment.

### DESCRIPTION OF THE EMBODIMENTS

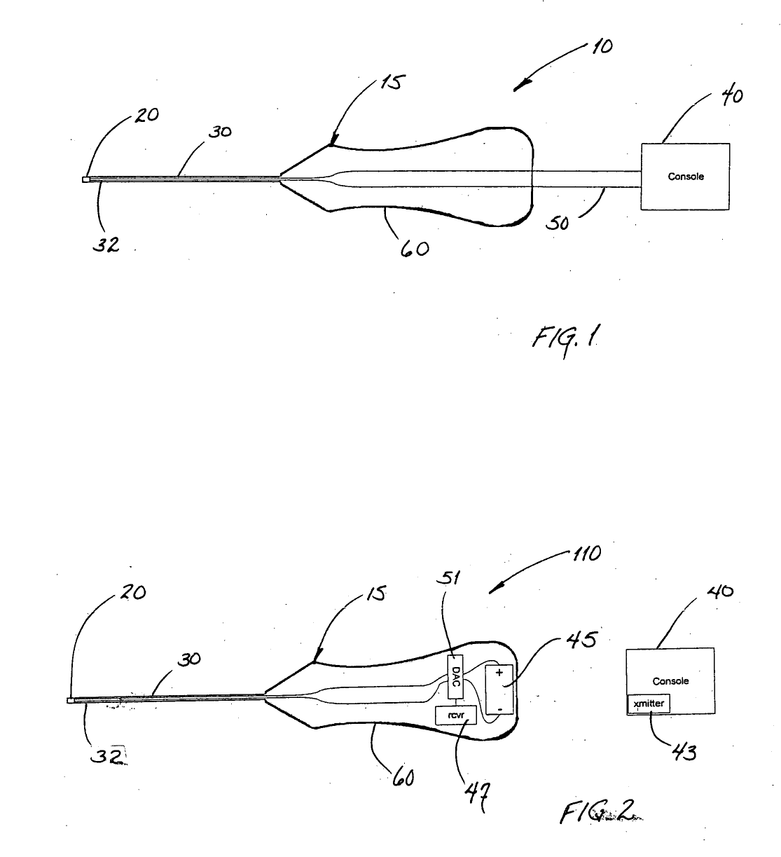

As may be seen by reference to FIGS. 1 and 2, the illustrated embodiments of the LED illuminator 10, 110 of the present invention provide an alternate method of illumination in which an LED 20 provides the source of illumination instead of the halogen lamp used in prior art illuminators. The small size and low operating temperature of presently available LED's enables the light source to be located on the end of the cannula 30 of the handheld probe assembly 15, as opposed to locating the light source in the console and conducting the light to the handpiece with a fiber optic cable. The end use functionality of the LED illuminator disclosed herein is at least as broad as the halogen illuminator. However, because the console assembly used with the disclosed LED illuminator is much lower in cost, and because the handheld probe assembly 15 is easier to manipulate due to the obviation of the light fiber from the console to the handheld probe 15, the disclosed LED illuminator will find greater utility than prior art halogen illuminators.

In the embodiments of the LED illuminator 10, 110 disclosed in FIGS. 1 and 2, the light emitting diode 20 is mounted directly at a first end 32 of the cannula 30. The first end 32 is sized and shaped depending on the size and shape of the space into which it is to be inserted. Such location enables placement of the illumination source in small cavities within the body of an animal, such as the posterior eye chamber, when the disclosed LED illuminator is used for a procedure within the animal such as eye surgery. The second end 34 of the cannula 30 provides for mounting in the hollow handle 60. As shown in FIG. 1, the electrical power to the LED 20 is provided from the console 40 via two thin wires 50. Alternatively, as shown in FIG. 2, electrical power may be provided by a battery 45 located in the handle 60 portion of the handheld probe assembly 15. When wires 50 are used between the console 40 and the handle 60, altering the current or electrical signal in the wires connecting the handle 60 to the console 40 controls the LED 20 brightness. In the wireless embodiment shown in FIG. 2, LED brightness would be controlled via a wireless interface between a transmitter 43 at the console 40 and a receiver 47 contained within the handle 60. Signals from the receiver 47 pass through a digital analog converter 51 on their way to the LED 20.

Because LED's are available with many different hues or color content, the use of different LED's is facilitated by the single use or disposable nature of the handheld probe assembly 15. Such flexibility allows for the use of an LED mounted in a small dome or a surface mount LED.

The LED illuminators 10, 110 described herein maintain the functionality of prior halogen lamp illuminators, but provide more flexibility to the user for probe manipulation and light filtering. If a disposable cannula 30 is used, a new LED “bulb” is provided for each use of the disposable handheld probe assembly 15.

The operation of the present invention is similar to prior art halogen light illuminators, with the exception of the setup procedure. For example, the LED illuminator of the present invention would require plugging in an electric cable connector, as opposed to the light pipe connector. The battery operated illuminator would need to be turned on by a switch located on the handle itself and then controlled by the console.

While the present invention has been disclosed according to the preferred embodiment of the invention, those of ordinary skill in the art will understand that other embodiments of the invention have also been enabled by the foregoing disclosure. Such other embodiments shall fall within the scope and meaning of the appended claims.

What is claimed is:

1. A system for illuminating an interior space within the body of an animal, said system comprising:
a cannula having a first end and a second end;
a hollow handle constructed and arranged for mounting said cannula;
said first end of said cannula being constructed and arranged for insertion into the interior space of the body of the animal;
said second end of said cannula being constructed and arranged for connection to said hollow handle;
said first end of said cannula further including a light emitting diode; and
means for providing an electrical signal for controlling the operation of said light emitting diode.

2. The system as defined in claim 1 wherein said means for providing an electrical signal are located in a console and said electrical signal is conducted to said light emitting diode by wires passing through said hollow handle and said cannula.

3. The system as defined in claim 1 wherein said means for providing an electrical signal is located in a console and said electrical signal is a wireless transmission to a receiver located in said hollow handle, which receiver is connected to said light emitting diode by wires passing through said hollow handle and said cannula.

4. The system as defined in claim 1 wherein the combination of said hollow handle, said cannula, and said light emitting diode are disposable.
